(19) United States
(12) Patent Application Publication
(10) Pub. No.: US 2005/0026149 A1
(43) Pub. Date: Feb. 3, 2005

(54) Nucleic acid constructs capable of high effeciency delivery of polynucleotides into dna containing organelles and methods of utilizing same

(76) Inventor: Ziv Reich, Ganei Yohanan (IL)

Correspondence Address: Anthony Castorina;G E Ehrlich, Suite 207, 2001 Jefferson Davis Highway, Arlington, VA, 22202, US

(21) Appl. No.: 10/488,936

(22) PCT Filed:
(86) PCT No.: PCT/IL02/00772

Related U.S. Application Data
(60) Provisional application No. 60/322,449, filed on Sep. 17, 2001.

(51) Int. Cl. C12Q 1/68; C07H 21/04; C12N 9/22; A61K 38/16

(52) U.S. Cl. 435/6; 435/69.1; 435/199; 435/320.1; 435/325; 530/358; 536/23.2

(57) ABSTRACT

A nucleic acid construct is provided. The nucleic acid construct includes: (a) a first polynucleotide segment including at least one nucleic acid sequence element; and (b) a second polynucleotide segment encoding a polypeptide including: (i) a nucleic acid binding domain being capable of specifically binding the at least one nucleic acid sequence element; and (ii) a localization signal for directing transport of the polypeptide into a DNA containing organelle, such that when the nucleic acid construct is introduced into a cell, expression of the polypeptide from the second polynucleotide segment directs transport of the nucleic acid construct into the DNA containing organelle.

### FIELD AND BACKGROUND OF THE INVENTION

The present invention relates to nucleic acid constructs capable of high efficiency delivery of polynucleotides into DNA containing organelles and methods of utilizing same.

Over the past 30 years, DNA delivery, especially via the nonviral route (i.e., transfection), has become a powerful and popular research tools for elucidating gene structure, regulation and function. DNA delivery has also been pivotal in developing new treatment approaches (e.g., gene therapy and DNA vaccination) and for treating and controlling diseases. However, before such applications can be realized, the relative inefficiency and cytotoxicity of modern DNA delivery systems must be addressed.

Viral Versus Non-Viral DNA Delivery Systems

Traditionally, DNA delivery systems have been classified as viral vector mediated systems and nonviral vector-mediated systems (i.e., synthetic systems). Due to their highly evolved and specialized components, viral systems are by far the most effective means of DNA delivery, enabling high efficiency in both delivery and expression. In fact, clinical protocols involving gene therapy typically utilize recombinant virus-based vectors for DNA delivery.

As yet, however, no definitive evidence has been presented for the clinical effectiveness of any gene therapy protocol [Anderson, W F. (1998) Nature 392:25-30]. Limitations of current viral based methodologies include toxicity, restricted targeting to specific cell types, limited DNA carrying capacity, production and packaging problems, recombination, and high costs [Crystal, R G. (1995) Science 270:404-410]. Due to these limitations, nonviral systems, especially synthetic DNA delivery systems, have found increasing use in both research laboratories and clinical settings.

Most synthetic DNA delivery systems operate at one of three general levels, DNA condensation and complexation, endocytosis, or nuclear targeting/entry. The negatively charged DNA molecules are usually condensed and/or complexed with cationic transfection reagents before delivery. These complexes are taken by cells, usually through endocytosis, with the route of uptake determining subsequent DNA release, trafficking and half life in the cell.

Endocytosis is a multistep process involving binding, internalization, formation of endosomes, fusion with lysosomes and lysis. The extremely low pH and presence of enzymes within the endosomes and lysosomes usually bring about degradation of entrapped DNA and associated complexes. DNA that has survived both endocytic processing and cytoplasmic nucleases must then dissociate from the condensed complexes either prior to or following entry into the nucleus. Entry occurs through nuclear pores, which are less than 10 nm in diameter. Once inside the nucleus, the transfection efficiency of delivered DNA is mostly dependent upon elements of the gene expression system.

This multistep process decreases the number of DNA molecules available at each step, thus resulting in an inefficient delivery of DNA into the nucleus.

DNA-Uptake

To date, most transformation methods concentrate on increasing DNA delivery through the plasma membrane barrier via physical or chemical approaches.

A. Mechanical and Electrical Approaches

(i) DNA microinjection—the direct injection of naked DNA (i.e., uncomplexed DNA) into a cell nucleus is perhaps the most conceptually simple and therefore appealing gene delivery approach. One drawback of this approach, however, is that microinjection can be achieved only one cell at a time, which limits its use to applications in which individual cell manipulation is desired and possible, such as producing transgenic organisms. Though relatively efficient, the method is rather slow and laborious and therefore not appropriate for large-scale applications.

(ii) Electroporation—this procedure uses high-voltage electrical pulses to transiently permeabilize cell membranes, thus permitting cellular uptake of macromolecules. Since it was first introduced [Wong, T K. and Neumann, C. (1982) Biochem. Biophys. Res. Commun. 107:584-587], electroporation has been used to deliver DNA into a myriad of cell types including bacteria and yeast. Although it is one of the most efficient gene transfer methods, high mortality of cells following high-voltage exposure and difficulties in optimization have limited use of this method.

(iii) Biolistic particle delivery—which is also termed particle bombardment is effected by accelerating DNA-coated microparticles, composed of metals such as gold or tungsten, to a velocity sufficient for penetrating cell membranes or cell walls [Yang, N S. (1990) Proc. Natl. Acad. Sci. 87:9568-9572]. Particle delivery is widely employed in DNA vaccination, where limited local expression of delivered DNA, such as in cells of the dermis or muscle, is adequate to achieve immune responses [Qiu, P. (1996) Gene Ther. 3:262-268]. However, because of the difficulty in controlling the DNA entry pathway, this approach is applied mainly on adherent cell cultures and has yet to be widely used systematically.

(iv) Micropricking—is an improved microinjection approach which utilizes needle-like bodies (such as glass or silicon carbide fiber) coated with DNA to impale cells and thus “inject” them with DNA (U.S. Pat. No. 5,464,765).

B. Chemical Methods

The general principle of DNA uptake-enhancing chemicals is based on complex formation between positively charged chemicals, typically polymers, and negatively charged DNA molecules.

(i) DEAE-Dextran and Calcium Phosphate methods—employ a high concentration of DEAE-dextran or Calcium phosphate, which are capable of interacting with DNA to form DEAE-dextran-DNA or Calcium phosphate-DNA complexes which are internalized by endocytosis. Although effective and simple, these methods are hampered by cytotoxicity and limited in-vivo applicability. In addition, DEAE-dextran cannot be used with culture medium which includes serum or for applications requiring stable transfection. The calcium phosphate method also suffers from variations in calcium phosphate-DNA sizes, which leads to inconsistent transformation efficiency.

(ii) Lipid based delivery systems—utilize artificial lipid-based DNA delivery systems, such as, Lipofectin, [Felgner, PL. (1987) Proc. Natl. Acad. Sci. USA 84: 7413-7417] which forms easily handled DNA complexes, and therefore was one of the first chemical systems used in animals. However, due to the poorly understood structure of DNA-lipid complexes, and variations arising during fabrication, such lipid based systems are of limited application.

Intracellular Routing of DNA

The Transformation efficiency can also be increased by protecting DNA from both extracellular and especially intracellular degradation.

Coating DNA with poly (ethylene glycol) (PEG) to form DNA capsules can be used to protect the DNA from degradation by nucleases [Lee, RJ. And Huang, L. (1997) Crit. Rev. Ther. Drug Carrier Syst.14:173-206]. It has been demonstrated that poly (L-Lyisine) (PLL)-g-PEG-DNA complexes are highly resistant to DNAase I attack [Katayose, S. and Kataoka, K. (1997) Bioconjug. Chem. 8:702-707]. Similar stabilization and protection of DNA has been achieved using PLL, epidermal growth factor (EGF) and streptavidin complexes in in-vitro transfection experiments. However, since most of these DNA protection methods involve complex formation with other molecules, liberation of DNA molecules from a macromolecular assembly must occur before transcription can proceed and therefore may affect efficiency of gene expression.

Nuclear Targeting

Movement of DNA through the cytosol toward the nucleus probably occurs by diffusion, a relatively slow process during which DNA is exposed to nucleases and the like. Certain synthetic polymers such as polyethylenimine (PEI), but not cationic lipids, protect DNA in the cytoplasm and are known to promote entry into the nucleus. In general, however, synthetic systems are notably more inefficient than viral vectors at targeting the nucleus.

(i) Nuclear localization signals (NLS)— Viral nuclear localization signals are a logical addition of synthetic DNA delivery systems. Indeed, fusion of an NLS peptide and a peptide nucleic acid (PNA) has been shown to facilitate nuclear transport of the PNA [Branden, J L. (1999) Nat. Biotechnol. 17:784-787]. Similarly, other fusion proteins, such as GAL4-NLS, have been employed to enhance transfection efficiency.

(ii) Nuclear targeting peptides— Most recently, nuclear-targeting peptide scaffolds have been conjugated and synthesized for lipid-based transfection of non-dividing mammalian cells [Subramanian, A. (1999) Nat. Biotechnol. 17:873-877]. Such scaffolds substantially enhance DNA delivery and gene expression. Although promising, this system is still restricted to nondividing cells and is yet to be extended to dividing cells.

Although advances in transformation methodology have led to an increase in transformation efficiency, currently employed approaches are still limited by cell type specificity, large-scale applicability, cytotoxicity, and laborious technical proceedings.

There is thus a widely recognized need for, and it would be highly advantageous to have, a highly efficient transformation approach devoid of the above limitations.

### SUMMARY OF THE INVENTION

According to one aspect of the present invention there is provided a nucleic acid construct comprising: (a) a first polynucleotide segment including at least one nucleic acid sequence element; and (b) a second polynucleotide segment encoding a polypeptide including: (i) a nucleic acid binding domain being capable of specifically binding the at least one nucleic acid sequence element; and (ii) a localization signal for directing transport of the polypeptide into a DNA containing organelle such that when the nucleic acid construct is introduced into a cell, expression of the polypeptide from the second polynucleotide segment directs transport of the nucleic acid construct into the DNA containing organelle.

According to another aspect of the present invention there is provided a nucleic acid construct comprising at least one nucleic acid sequence element being selected such that when the nucleic acid construct is introduced into a eukaryotic cell endogenously expressing a polypeptide including: (a) a nucleic acid binding domain being capable of binding the nucleic acid sequence element; and (b) a localization signal for directing transport of the polypeptide into a DNA containing organelle; the nucleic acid construct actively transports into the DNA containing organelle.

According to yet another aspect of the present invention there is provided a method of facilitating active transport of a polynucleotide of interest into a DNA containing organelle of a eukaryotic cell, the method comprising: (a) introducing into a cytoplasm of the eukaryotic cell a nucleic acid construct including the polynucleotide of interest and at least one nucleic acid sequence element; and (b) providing within the cytoplasm of the eukaryotic cell a polypeptide including: (i) a nucleic acid binding domain being capable of specifically binding the at least one nucleic acid sequence element; (ii) a localization signal for directing transport of the polypeptide into the DNA containing organelle thereby facilitating active transport of the polynucleotide of interest into the DNA containing organelle of the eukaryotic cell.

According to still further features in the described preferred embodiments the step of providing within the eukaryotic cell the polypeptide is effected by a lipid based delivery system.

According to further features in preferred embodiments of the invention described below, the polypeptide is endogenous to the eukaryotic cell and further wherein the step of providing within the eukaryotic cell the polypeptide is effected by inducing expression or activity of the polypeptide.

According to still further features in the described preferred embodiments the step of providing within the eukaryotic cell the polypeptide is effected by introducing into the eukaryotic cell an additional nucleic acid construct capable of expressing the polypeptide.

According to still another aspect of the present invention there is provided a nucleic acid construct system comprising: (a) a first nucleic acid construct including at least one nucleic acid sequence element; and (b) a second nucleic acid construct including a polynucleotide segment encoding a polypeptide including: (i) a nucleic acid binding domain being capable of specifically binding the at least one nucleic acid sequence element; (ii) a localization signal for directing transport of the polypeptide into a DNA containing organelle; such that when the first and the second nucleic acid constructs are introduced into a cell, expression of the polypeptide from the polynucleotide segment of the second nucleic acid construct directs transport of the first nucleic acid construct into the DNA containing organelle.

According to an additional aspect of the present invention there is provided a method of detecting and isolating polynucleotides encoding polypeptides capable of binding a nucleic acid sequence element of interest, the method comprising: (a) preparing a library of nucleic acid constructs each including: (i) a first polynucleotide segment including at least one nucleic acid sequence element; (ii) a second polynucleotide segment capable of generating reporter activity; (iii) a third polynucleotide segment encoding a chimeric polypeptide including a distinct putative nucleic acid sequence binding domain and a localization signal for a DNA containing organelle; (b) introducing the expression library into a plurality of eukaryotic cells; and (c) screening for a cell or cells of the plurality of cells exhibiting a predetermined level or localization pattern of the reporter activity, thereby detecting and isolating polynucleotides encoding polypeptides capable of binding the nucleic acid sequence element of interest.

According to yet an additional aspect of the present invention there is provided a method of detecting and isolating nucleic acid sequence elements, being bound by a polypeptide of interest, the method comprising:(a) preparing a library of nucleic acid constructs each including: (i) a first polynucleotide segment including a distinct putative nucleic acid sequence element; (ii) a second polynucleotide segment capable of generating reporter activity; (iii) a third polynucleotide segment encoding a chimeric polypeptide including the polypeptide of interest and a localization signal for a DNA containing organelle; (b) introducing the expression library into a plurality of eukaryotic cells; and (c) screening for a cell or cells of the plurality of cells exhibiting a predetermined level or localization pattern of the reporter activity, thereby detecting and isolating nucleic acid sequence elements being bound by the polypeptide of interest.

According to further features in preferred embodiments of the invention described below, the DNA containing organelle is selected from the group consisting of a nucleus, a chloroplast and a mitochondria.

According to still further features in the described preferred embodiments the third polynucleotide segment is positioned under a transcriptional control of the at least one nucleic acid sequence element.

According to still further features in the described preferred embodiments the at least one nucleic acid sequence element is as set forth in SEQ ID NO:3.

According to still further features in the described preferred embodiments the localization signal for the DNA containing organelle is selected from the group consisting of a nuclear localization signal (NLS), a mitochondrial localization signal (MLS) and a chloroplast localization signal (CLS).

According to still further features in the described preferred embodiments the first polynucleotide region is positioned upstream or downstream of the second polynucleotide region.

According to still further features in the described preferred embodiments the at least one nucleic acid binding domain is derived from a nucleic acid binding protein.

According to still further features in the described preferred embodiments the nucleic acid binding protein is a transcription factor.

According to still further features in the described preferred embodiments the eukaryotic cell comprising the nucleic acid construct.

The present invention successfully addresses the shortcomings of the presently known approaches by providing nucleic acid constructs capable of high efficiency delivery of polynucleotide sequences into DNA containing organelles.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic illustration of a κB-pGL3 expression construct. κB-pGL3 was generated on pGL3 backbone and includes: five κB sites (5×κB, SEQ ID NO:1), ten PNA binding sites (PNA, SEQ ID NO:2), an SV40 promoter (SV40P), a Luciferase coding sequence (Luc), a polyadenylation site (poly A), an SV40 enhancer (SV40E), an ampicillin selectable marker gene (Amp).

FIGS. 2a-d illustrates transfection efficiency of pGL3 and κB-pGL3 as determined by a Luciferase assay. The human cell lines HeLa (FIG. 2a), Hek-293 (FIG. 2b), HepG2 (FIG. 2c) and U373 (FIG. 2d) were transiently transfected with a control vector pGL3 (open bars) or with κB-pGL3 (closed bars). Following transfection, TNF-α was added to the transfected cells, as specified, and cells were harvested for analysis 18 hours later. Luciferase activity was measured, and was normalized to total protein or β-galactosidase activity. The increase in Luciferase activity resulting from TNF-α treatment is indicated by fold induction as determined relative to nonstimulated cells transfected with pGL3.

FIGS. 3a-d illustrates nuclear import of control and modified pGL3 as monitored by confocal fluorescence microscopy. HeLa cells were incubated for 10 hours with 1 μg of rhodamine-labeled pGL3 (FIGS. 3a-b) or with κB-pGL3 (FIGS. 3c-d), in the absence (FIGS. 3a-c) or presence (FIGS. 3b-d) of TNF-α. Subsequently, cells were fixed and visualized with an Olympus Fluoview 200 confocal laser-scanning microscope (BX50WI-based), using a 60×PlanApo immersion objective (NA 1.4). The deep blue of the central nuclear regions depicted in (FIG. 3a) represents background fluorescence slightly higher than that exhibited by nontransfected cells.

FIGS. 4a-b illustrates the transcriptional contribution of κB elements. HeLa cells were transfected (0.5 μg DNA/2·10⁵ cells) with control and modified pGL3 (FIG. 4a) or NFκB-Luc (FIG. 4b) and were stimulated with TNF-α for 5 hr prior to harvesting. Luciferase activity was measured, and normalized to total protein or β-galactosidase activity. The increase in Luciferase activity resulting from TNF-α treatment is indicated by fold induction relative to nonstimulated cells.

### DETAILED DESCRIPTION OF THE PREFERRED EMBODIMENTS

The present invention is of nucleic acid constructs and methods utilizing same, which can be used to facilitate transformation of eukaryotic cells. Specifically, the present invention can be used to direct active transport of nucleic acid sequences into DNA containing organelles. In addition, the present invention can also be used to isolate novel genes encoding nucleic acid sequence binding proteins.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings described in the Examples section. The invention is capable of other embodiments or of being practiced or carried out in various ways. Also, it is to be understood that the phraseology and terminology employed herein is for the purpose of description and should not be regarded as limiting.

The ability to safely and efficiently transfer DNA into cells is a fundamental goal in biotechnology. Current synthetic DNA delivery systems, although safe, are relatively inefficient. One of the major obstacles to efficient gene-delivery is targeting the genetic material into the DNA containing organelle, such as the nucleus. In current gene delivery methods, movement of DNA through the cytosol toward the nucleus occurs via diffusion, a relatively slow process during which the genetic material is exposed to a degrading cytoplasmic environment.

The present invention provides a novel approach for facilitating active uptake of polynucleotide sequences into a DNA containing organelle, such as the nucleus. As described hereinunder and in the Examples section which follows, the present invention provides novel nucleic acid constructs capable of active and controlled delivery of polynucleotides into a variety of DNA containing organelles and as such can be used to efficiently transform cells even in cases in which polynucleotide quantities are limited or in cases where nuclear entry is limited, such as the case with large DNA constructs.

Thus, according to one aspect of the present invention there is provided a nucleic acid construct capable of being actively transported into a DNA containing organelle.

The phrase “DNA containing organelle” refers to a specific, usually subcellular, membrane-encapsulated structure, present in all eukaryotic cells. DNA containing organelles include, the mitochondrion, the nucleus, the chloroplast, the proplast, the etioplast, the chromoplast and the leukoplast, and any subcellular structure which includes endogenous DNA molecules.

The nucleic acid construct of the present invention includes at least one nucleic acid sequence element which is selected such that when the nucleic acid construct of the present invention is introduced into the cytoplasm of a eukaryotic cell expressing a polypeptide including: (i) a nucleic acid binding domain being capable of specifically binding the at least one nucleic acid sequence element and (ii) a localization signal for directing transport of the polypeptide into a DNA containing organelle, the nucleic acid construct is actively transported into the DNA containing organelle.

The nucleic acid construct can be introduced into the cell via any transformation method known in the art. The Background and Examples section herein provide description of various transformation methods suitable for use with the present invention.

As used herein, the phrase “nucleic acid sequence element” refers to a segment anywhere between 3 to 500 nucleotides long, which is capable of forming a secondary and/or tertiary structure which specifically interacts with a nucleic acid binding domain of a nucleic acid binding protein.

Examples of nucleic acid sequence elements include promoter sequences which are capable of binding with transcriptional activators or regulatory sequences, such as the lgK κB sequence element set forth in SEQ ID NO:3, which interacts with the NFκB transcription regulator.

Alternatively a nucleic acid sequence element can be a translational regulatory element which specifically binds with a translational regulatory polypeptide and functions in regulating translation.

Nucleic acid sequence elements can include various sequence motifs such as, but not limited to, direct repeats, palindromes, inverted palindromes and isolated half-sites. Typically, the binding sites of nucleic acid sequence elements are less than 20 nucleotides in length although multiple binding sites may be positioned adjacent to each other in a single nucleic acid sequence element.

As mentioned hereinabove, the nucleic acid construct is introduced into a cell provided with a polypeptide capable of binding the nucleic acid construct of the present invention and actively transporting it into a DNA containing organelle. To enable specific polypeptide-construct interaction, the polypeptide described hereinabove includes a nucleic acid sequence binding domain which is capable of specifically binding with the nucleic acid sequence element. The polypeptide further includes a localization signal for self transport into the DNA containing organelle.

The localization signal can be for example, a nuclear localization signal (NLS), such as a short predominantly basic amino acid sequence, which is recognized by specific receptors at the nuclear pores. The localization signal for a DNA containing organelle can also be a mitochondrial localization signal (MLS) or a chloroplast localization signal (CLS).

According to one preferred embodiment of the present invention, the polypeptide is endogenous to the cell transformed. Numerous cell types express endogenous polypeptides suitable for use with the present invention and as such, can be efficiently transformed with the nucleic acid construct of the present invention.

Preferably, the endogenously expressed nucleic acid binding polypeptide can translocate to the DNA containing organelle following induction.

Examples of such endogenous polypeptides are the members of the NFκB/Rel family (described in the Examples section, which follows) and the steroid receptor family.

Translocation of the polypeptide into the DNA containing organelle can be induced by a factor such as a growth regulating factor, a cytokine, a hormone, a lymphokine, a steroid, a neurotransmitter, a chemotaxin, an antigen or a toxin.

Alternatively, induction can be effected via a physical stimulus such as, but not limited to, irradiation, for example, X-irradiation, UV irradiation and gamma-irradiation. A physical stimulus can also be light conditions and temperature conditions.

According to another preferred embodiment of the present invention, the polypeptide described hereinabove can be exogenous to the cell transformed.

An exogenous polypeptide can be any of the polypeptide types described hereinabove (e.g., transcriptional factors) or it can be a chimeric polypeptide which includes a localization signal for transport into a DNA containing organelle and a nucleic acid binding domain.

As used herein the phrase “chimeric polypeptide” refers to a polypeptide fusion in which sequences from two or more polypeptides are linked via peptide bonds.

In the chimeric polypeptide, the nucleic acid binding domain can be derived, for example, from a DNA binding protein (e.g., histones) or from proteins which regulate gene expression (e.g., transcriptional factors). Such domains can be selected so as to have any of the following DNA binding motifs, helix-turn-helix, homeodomains, zinc-finger, steroid receptor, beta sheets, leucine zipper, helix-loop-helix and the like. Further detail on DNA binding domains is provided by Faisst, S., and Meyer S., (1992) Nucl. Acids Res. 20:3-26.

Alternatively, the nucleic acid binding domain can be a portion of an RNA binding protein (RBP), in which case, the nucleic acid construct used for transformation would be an RNA construct. In this case such domains are selected so as to have any of the following motifs: an RNP motif, an Arg-rich motif, an RGG box, a KH motif and a double-stranded RNA-binding motif [Burd and Dreyfuss (1994) Science 265:615-621].

In any case, one skilled in the art will appreciate that any portion of a nucleic acid sequence binding domain can be utilized as long as high affinity binding to the corresponding nucleic acid sequence element is retained.

The exogenous polypeptide can be expressed from the nucleic acid construct described hereinabove, or from an additional nucleic acid construct which is co-introduced therewith into the cell.

Alternatively, the exogenous polypeptide can be introduced into the cell as a polypeptide, via for example, liposome delivery [Uchimiya, H.et al., (1982) Cong. Plant Tissue and Cell Culture, Jap. Assoc. for Plant Tissue Culture, Tokyo,. 507-50], peptide microinjection [Piacumakos, E G., (1973) Methods Cell Biol. 287-311], micropricking [Yamamoto F., (1982) Exp. Cell Res. 142:79-84] or ionophoresis [Purres R D., (1981) Acad. Press NY. 146].

In such cases, the polypeptide can be expressed and collected from another cell system as a native polypeptide, or it can be synthesized in-vitro via well known prior art methods.

A synthetic polypeptide can include modifications rendering the polypeptide more stable while in the cell. Such modifications include, but are not limited to N teiminus modification, C terminus modification, peptide bond modification, including, but not limited to, CH2—NH, CH2—S, CH2—S═O, O═C—NH, CH2—O, CH2—CH2, S═C—NH, CH═CH or CF═CH, backbone modifications, and residue modification. Methods for preparing modified polypeptides are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the polypeptide may be substituted, for example, by N-methylated bonds (—N(CH3)—CO—), ester bonds (—C(R)H—C—O—O—C(R)—N—), ketomethylen bonds (—CO—CH2—), α-aza bonds (—NH—N(R)—CO—), wherein R is any alkyl, e.g., methyl, carba bonds (—CH2—NH—), hydroxyethylene bonds (—CH(OH)—CH2—), thioamide bonds (—CS—NH—), olefinic double bonds (—CH═CH—), retro amide bonds (—NH—CO—), peptide derivatives (—N(R)—CH2—CO—), wherein R is the “normal” side chain, naturally presented on the carbon atom.

These modifications can occur at any of the bonds along the polypeptide chain and even at several (2-3) at the same time.

Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

In addition to the above, the polypeptides of the present invention may also include one or more modified amino acids or one or more non-amino acid monomers (e.g. fatty acids, complex carbohydrates etc).

As used herein the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables. 1 and 2 below list naturally occurring amino acids (Table 1) and non-conventional or modified amino acids (Table 2) which can be used with the present invention.

TABLE 1

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Iie | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 2

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N- amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | Penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | Penicillamine | Pen |
| L-homophenylalanine | Hphe | L-α-methylalanine | Mala |
| L-α--methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α--methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval Nnbhm | L-N-methylhomophenylalanine | Nmhphe |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| ethylamino)cyclopropane | | | |

The nucleic acid constructs of the present invention preferably also include an appropriate selectable marker and an origin of replication in bacteria.

The nucleic acid constructs can be constructed using commercially available eukaryotic expression vectors or derivatives thereof. Examples of suitable vectors include, but are not limited to pcDNA3, pcDNA3.1 (+/−), pGL3, PzeoSV2 (+/−), pSecTag2, pDisplay, pEF/myc/cyto, pCMV/myc/cyto, pDR3.1, pSinRep5, DH26S, DHBB, pNMT1, pNMT41, pNMT81, which are available from Invitrogen, pMbac, pPbac, pBK-RSV and pBK-CMV which are available from Stratagene, pTRES which is available from Clontech.

Any of the promoter and/or regulatory sequences included in the expression vectors described above can be utilized to direct the transcription of the exogenous polypeptide, described hereinabove.

Preferably, the promoter that is selected according to the host cells or tissues of interest. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166). Promoters for expression of the polynucleotide can also be developmentally-regulated promoters such as the murine homeobox promoters [Kessel et al. (1990) Science 249:374-379) or the α-fetoprotein promoter [Campes et al. (1989) Genes Dev. 3:537-546].

The nucleic acid constructs of the present invention may also include one or more polynucleotides of interest.

In cases where the nucleic acid sequence element is derived from a transcriptional regulatory sequence, the polynucleotide(s) of interest is preferably positioned in proximity to the nucleic acid sequence element thus also providing transcriptional upregulation of the polynucleotide of interest.

The polynucleotide(s) of interest can be any sequence which benefits from high efficiency transport into a DNA containing organelle. Thus, the polynucleotide(s) of interest can be, for example, a protein encoding sequence, a functional RNA encoding sequence, or gene knock-in/out elements.

Although the present invention, as described herein above, is aimed at transformation of cultured cells, it may also be used for tissue and whole organism gene delivery as part of, for example, a gene therapy procedure.

In such cases, the nucleic acid construct of the present invention also includes a polynucleotide of interest encoding a therapeutic RNA molecule or protein. Such a nucleic acid construct is preferably administered as part of a composition (e.g., with a physiological carrier, such as a pharmaceutically acceptable carrier), using well known administration routes which are selected suitable for a particular application.

The nucleic acid construct can be provided in a unit dosage form for administration in the context of the present inventive method, wherein each dosage unit, e.g., a solution, contains a predetermined amount of the nucleic acid construct, alone or in appropriate combination with other active agents, such as the transport polypeptide, discussed hereinabove.

The term “unit dosage form” as used herein refers to physically discrete units suitable as unitary dosages for human and animal subjects, each unit containing a predetermined quantity of the exogenous nucleic acid of the present invention, alone or in combination with other active agents, calculated in an amount sufficient to produce the desired effect, in association with a physiological carrier (e.g., a pharmaceutically acceptable carrier) where appropriate. The specifications for the unit dosage forms depend on the particular effect to be achieved and the particular pharmacodynamics associated with the exogenous nucleic acid composition in the particular host.

The “effective amount” of the exogenous nucleic acid composition is such as to produce the desired effect in a host that can be monitored using several end-points known to those skilled in the art. Furthermore, the preferred amounts of the nucleic acid construct of the present invention (e.g., per cell) ranges from about 1 to about 150 μg plasmid DNA, although amounts below 1 μg plasmid DNA and above 150 μg can also be used by the present invention. The actual dose and administration schedule can vary depending on whether the exogenous nucleic acid is administered in combination with other active agents, or depending on inter-individual differences in pharmacokinetics, drug disposition, and metabolism. Furthermore, the amount of the nucleic acid construct to be administered will likely vary with the length and stability of the polynucleotide of interest, as well as the nature of the sequence.

Thus, the present invention provides a novel approach for increasing polynucleotide transport into a DNA containing organelle thereby enhancing any DNA transformation approach, including DNA vaccination approaches designed for therapeutic purposes.

According to another aspect of the present invention there is provided a method of detecting and isolating polynucleotides encoding polypeptides capable of binding a nucleic acid sequence element of interest.

The method according to this aspect of the present invention is effected by first preparing an expression library of a plurality of expression constructs each including at least one copy of the nucleic acid sequence element described above, and a polynucleotide capable of generating a reporter activity. Such reporter activity can be provided by, for example, a PNA-peptide nucleic acid labeling site present within this polynucleotide (see Example 3 of the Examples section below), or by a reporter molecule expressed from this polynucleotide (e.g., GFP, Misawa et al. (2000) Proc. Natl, Acad. Sci. 97:3062-3066).

Each of the expression constructs further includes a polynucleotide segment which encodes a chimeric polypeptide including a putative nucleic acid sequence binding domain and a localization signal for transport into a DNA containing organelle.

The putative binding domain can be encoded by cDNA fragments obtained by reverse transcribing and optionally PCR amplifying mRNA isolated from any one or more cells, tissues or organisms, it can be a synthetic nucleic acid, a fragmented nucleic acid derived from a genome or a combinatorial nucleic acid. The putative binding site can be relatively short, including several amino acids, or it can be long, including several hundred amino acids.

Each of the expression constructs of the expression library of the present invention further includes at least one cis acting regulatory element, e.g., a promoter and an enhancer, for directing expression of the chimeric polypeptide. It will be appreciated by one skilled in the art that in the process of preparation of a library as herein described many individual constructs may be inoperative due to, for example, out of frame ligations, etc. This however, can be readily overcome by increasing library size, so as to have sufficient representation of operative constructs.

The library constructs according to the present invention preferably further include an appropriate selectable marker and an origin of replication, as discussed above.

Following library generation, the expression library is introduced into a plurality of eukaryotic cells. Measures are taken so as to control the number of constructs entering a particular cell; preferably a single construct is introduced to an individual cell.

Following some time in culture the transformed cells are manually or automatically screened for a cell or cells in which the reporter activity follows a specific pattern (e.g., predominantly, or significantly more, localized to the DNA containing organelle as compared with control cells) or in which reporter activity is increased as a result of enhanced nuclear entry and thus enhanced reporter molecule expression.

The polynucleotides encoding the putative nucleic acid sequence binding polypeptide are isolated from such cells via PCR amplification or similar techniques. PCR amplification of the polynucleotide of interest can be readily effected because the sequences flanking the polynucleotide are known and suitable amplification primers can therefore be designed. Procedures for effecting PCR amplification as described herein are described in, for example, “PCR protocols: A Guide to Methods And Applications”, Academic Press, San Diego, Calif. (1990).

Further confirmation of the putative nucleic acid binding domain can be effected using prior art methods such as EMSA, south-western blots, overlay assays and the like. Once identified, cells that are of interest for further study are stored frozen.

It will be appreciated that the above described method can also be utilized to uncover novel nucleic acid sequence elements by generating a library of constructs each including a putative nucleic acid sequence element and a reporter polynucleotide and introducing this library into cells expressing a known transport polypeptide (e.g., the NF κB described above).

Additional objects, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion. Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorpotaed by reference as if fully set forth herein.

### Example 1

### Construction of κB-pGL3 Expression Vector

Synthetic (nonviral) gene delivery systems are promising tools for gene therapy and DNA vaccination applications. Compared to viral-based systems, they possess several advantages including safety profiles, an essentially unlimited DNA carrying capacity and ease of production. However, transfection efficiency by these methods is severely low, largely due to the inability of the DNA to effectively translocate through the nuclear pore complexes (NPCs).

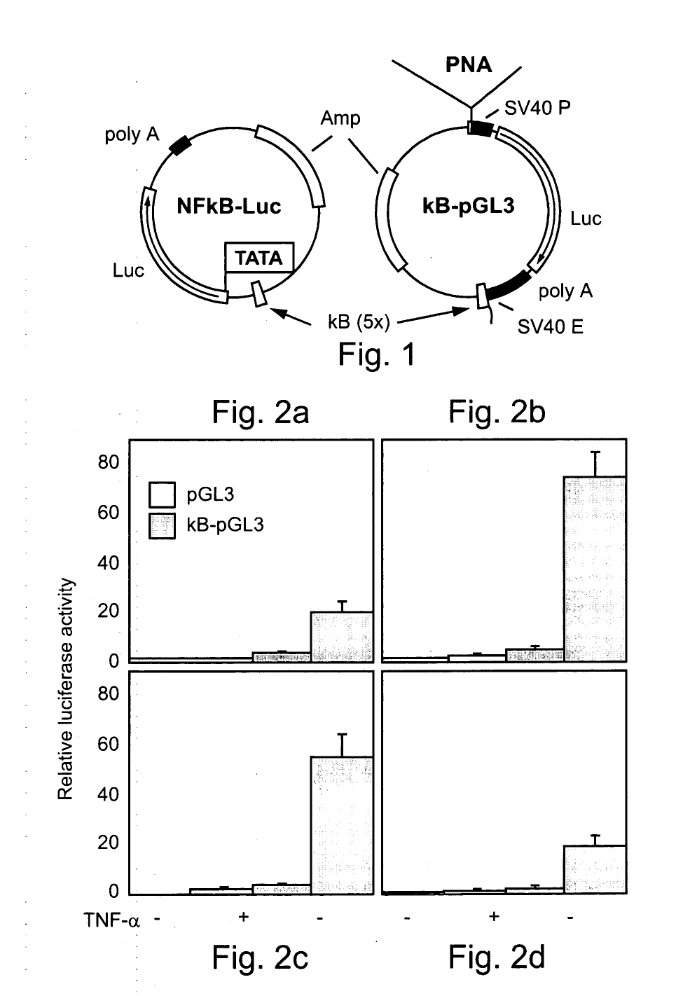

FIG. 1 outlines an expression construct of the present invention which can be used for facilitating delivery of polynucleotides into the nucleus.

The expression construct, termed κB-pGL3, was generated by cloning a PCR amplified fragment, including five tandem repeats of the κB motif (SEQ ID NO:1) into the pGL3 vector (Promega). The κB motif serves as a high affinity (K_{D}˜10⁻¹⁰−10−¹³ M) binding site for the rapid response transcription factors, nuclear factor κB (NF κB)/Rel family (p49, p50 and p65) allowing them to bind the DNA in the cytoplasm and transport it to the nucleus through the protein nuclear import machinery, following stimulation.

κB-pGL3 includes an SV40 promoter and enhancer elements for directing expression of a polynucleotide of interest in eukaryotic cells. The κB sites were cloned downstream of the SV40 enhancer in κB-pGL3 so as to ensure that the κB sites do not participate directly in the transcriptional regulation of the Luciferase cloned transgene (GenBank accesion number U47298.2), operably linked to the SV40 promoter.

To enable fluorescence labeling of pGL3 and κB-pGL3, a segment containing peptide nucleic acid (PNA) recognition sequence (SEQ ID NO:2) was excised (BsaI/BgIII) from pGeneGrip (Gene Therapy Systems) and cloned in the multicloning sites region (BgIII/SmaI) of the κB-pGL3 modified plasmid. Modifications were confirmed by double-stranded sequencing. Plasmid DNA was purified on Qiagen maxiprep-columns cleaned with phenol/chloroform, ethanol precipitated and stored frozen.

### Example 2

### Elevated Transgene Expression Mediated by κB-pGL3

To test the ability of the expression constructs of the present invention to elevate transgene expression, transfection efficiencies of the control vector, pGL3 and κB-pGL3 were assayed using Luciferase activity as a functional measure (FIGS. 2a-d).

The human cell lines HeLa, Hek-293, HepG2 and U373, all responsive to known NF κB stimuli such as, tumor necrosis factor-α (TNF-ε) and TPA, were grown in Dulbecco's Modified Eagle's medium (DMEM) supplemented with 10% fetal calf serum (FCS), and subsequently serum starved 6 hours prior to transfection. Thereafter, cells were transfected with 0.25 μg of the appropriate construct using SuperFect (Qiagen). Following transfection, rhTNF-α (20 ng/ml Pepro Tech EC) was added to the transfected cells, as specified, and cells were harvested for analysis 18 hours later. Luciferase activity was measured with the Luciferase Assay System (Promega) and was normalized to total protein (Pierce) or β-galactosidase activity (Promega). Data was generated from at least three independent measures; SEM values were calculated using compound quantity formulation.

As shown in FIGS. 2a-d, Luciferase activity measured in extracts of κB-pGL3 transfected cells was substantially higher than that measured from extracts of pGL3 transfected cells following TNF-α stimulation. Moreover, in the absence of TNF-α stimulation κB-pGL3 transfected cells still displayed elevated Luciferase activity as compared to pGL3 transfected cells, suggesting a low-level migration of NF κB molecules into the nucleus. The minor increase in Luciferase activity that was observed in TNF-α stimulated pGL3-expressing cells could be explained by the presence of an intrinsic κB site in its SV40 enhancer region.

In conclusion the data presented hereinabove suggest that the expression construct of the present invention facilitates elevated transgene expression, and therefore is a useful research and clinical tool.

### Example 3

### Controlled Nuclear Targeting of κB-pGL3

The subcellular distribution of NF κB is controlled by a family of inhibitory proteins, I κBs, which bind NF κB and mask its nuclear localization signal, thereby preventing nuclear uptake. Exposure of cells to a variety of extracellular stimuli leads to the rapid degradation of I κB, which releases NF κB to translocate to the nucleus, where it regulates gene transcription. The construct described in Example 1 above utilizes the ability of NF κB to bind DNA through κB sequence elements and to translocate into the nucleus following stimulation, in order to facilitate active transport of polynucleotide sequences into the nucleus.

To determine the efficiency of NF κB mediated transport, nuclear transport of rhodamine-labeled pGL3 and κB-pGL3 was determined via confocal microscopy. HeLa cells (10⁵ cells/sample) were loaded with rhodamine-labeled plasmids (1 μg for 10 hours) in the presence or absence of TNF-α. Labeling of plasmids was effected via site-specific hybridization to rhodamine-labeled PNA clamps (Gene Therapy Systems) PNA clamp labeling preserves the plasmid's native supercoiled conformation while it does not alter nuclease sensitivity and intracellular distribution thereof [Zelphati O et al. (1999) Gene Chemistry 10:15-24]. Moreover, since binding is sequence-specific and highly stable, the fluorophore content on each plasmid molecule is identical and undesired labeling of endogenous nucleic acids is avoided.

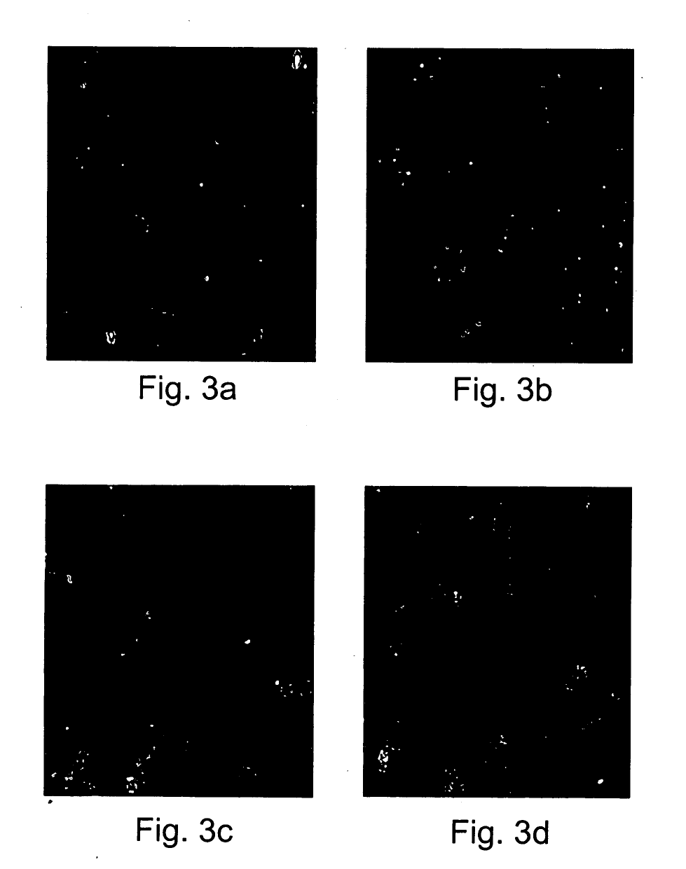

Following loading and stimulation, cells were fixed (4% paraformaldehyde/5% sucrose, in PBS) and visualized with an Olympus Fluoview 200 confocal laser scanning microscope (BX50WI-based) using a 60X PlanApo immersion objective (NA 1.4). Data processing was effected using the NIH Image software. The resulting images are presented in FIGS. 3a-d and are representative of ˜200 images acquired from 3 independent experiments.

As illustrated in FIGS. 3a-d transfection of cells with the pGL3 plasmid resulted in punctate cytoplasmic fluorescence and low nuclear signal (FIG. 3a); further stimulation with TNF-α did not significantly alter the fluorescence pattern (FIG. 3b). On the other hand, cells transfected with κB-pGL3 exhibited clear nuclear fluorescence with a diffused appearance, while cytoplasmic staining was significantly reduced (FIG. 3c). Furthermore, application of TNF-α caused a marked increase in nuclear fluorescence, while cytoplasmic distribution was barely detectable.

Thus, the results obtained herein provide direct evidence that κB-pGL3 translocation to the nucleus can be regulated by TNF-α.

### Example 4

### κB Sequence Elements Can Serve as Transcriptional Enhancers

The expression of nuclear genes is controlled by interactions between the upstream promoter and regulatory factors that bind to specific DNA elements. Basal levels of gene expression are controlled by core promoter elements such as Spl, CAAT or TATA boxes. These are short DNA sequences that define the position within the gene at which transcription is initiated [Williams R S. (1990) Circulation 82:319-331]. Changes in transcription rates in response to physiological signals are mediated by transcription factors, which reversibly bind with specific nucleotide sequences within the promoter (regulatory elements). These regulatory elements can be located hundreds of nucleotides up- or downstream of the transcription initiation site and may be either stimulatory (enhancers) or inhibitory (repressors/suppressors). The responsiveness of the gene is determined by the number, type and organization of regulatory elements within promoter regions.

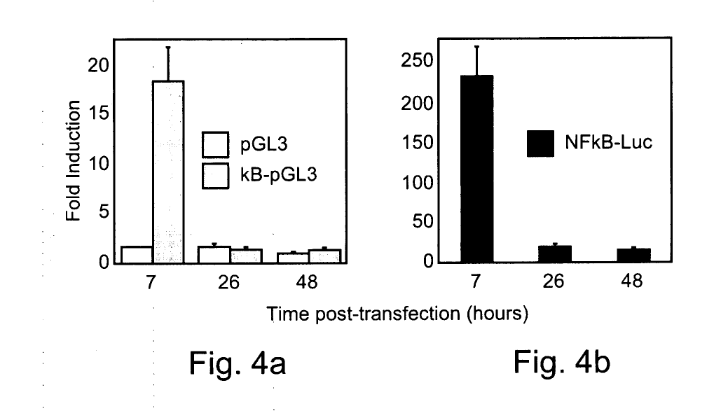

To quantify the contribution of the κB sites to transcription of the reporter molecule, a time course study was performed using control and modified pGL3 (illustrated in FIG. 1), as well as pNFκB-Luc (Stratagene). Unlike the κB-pGL3 where the κB sites were subcloned downstream of the Luciferase open reading frame, in pNFκB-Luc the κB sites were subcloned adjacent to the TATA box, so as to support enhanced transcriptional activity. HeLa cells were transfected with each of the plasmids and incubated for 2, 24, or 43 hours. Cells were stimulated at the end of each incubation period with TNF-α (20 ng/ml). Starting at 5 hours, cells were harvested and analyzed for Luciferase activity. As is illustrated in FIG. 4a-b, at 7 hours post-transfection Luciferase generated by both pNFκB-Luc and pGL3-κB was significantly higher than hat generated from the control pGL3 vector. Interestingly, pNFκB-Luc transfected cells (FIG. 4b) exhibited far higher Luciferase activity (over 20 fold) as compared to pGL3-κB expressing cells (FIG. 4a), suggesting that the κB sequence elements may have an additional transcriptional enhancer activity. This was substantiated by the observations that at twenty-six hours and at forty-eight hours post-transfection, Luciferase activity generated by pNFκB-Luc was markedly increased than that generated by pGL3 and its derivative form pGL3-κB (1.3 and 1.6 fold induction, respectively). Within these time intervals, at least one cell division could take place, leading to similar levels of nuclear accumulation of the plasmids, due to nuclear envelope breakdown concomitant with mitosis. Thus the increase in gene activity, observed upon stimulation, should reflect primarily contributions arising from transcriptional enhancement by the κB sites in the pNFκB-Luc vector.

These results suggest that the system presented herein can provide a control element which is effective at both nuclear uptake and transcription enhancement.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

1. A nucleic acid construct comprising:
(a) a first polynucleotide segment including at least one nucleic acid sequence element; and
(b) a second polynucleotide segment encoding a polypeptide including:
(i) a nucleic acid binding domain being capable of specifically binding said at least one nucleic acid sequence element; and
(ii) a localization signal for directing transport of said polypeptide into a DNA containing organelle
such that when the nucleic acid construct is introduced into a cell, expression of said polypeptide from said second polynucleotide segment directs transport of the nucleic acid construct into said DNA containing organelle.

2. The nucleic acid construct of claim 1, wherein said DNA containing organelle is selected from the group consisting of a nucleus, a chloroplast and a mitochondria.

3. The nucleic acid construct of claim 1, wherein said at least one nucleic acid binding domain is derived from a nucleic acid binding protein.

4. The nucleic acid construct of claim 3, wherein said nucleic acid binding protein is a transcription factor.

5. A eukaryotic cell comprising the nucleic acid construct of claim 1.

6. The nucleic acid construct of claim 1, further comprising a third polynucleotide segment encoding a second polypeptide.

7. The nucleic acid construct of claim 6, wherein said third polynucleotide segment is positioned under a transcriptional control of said at least one nucleic acid sequence element.

8. The nucleic acid construct of claim 1, wherein said at least one nucleic acid sequence element is as set forth in SEQ ID NO:3.

9. The nucleic acid construct of claim 1, wherein said a localization signal for directing transport of said polypeptide into said DNA containing organelle is selected from the group consisting of a nuclear localization signal (NLS), a mitochondrial localization signal (MLS) and a chlioroplast localization signal (CLS).

10. A nucleic acid construct comprising at least one nucleic acid sequence element being selected such that when the nucleic acid construct is introduced into a eukaryotic cell endogenously expressing a polypeptide including:
(a) a nucleic acid binding domain being capable of binding said nucleic acid sequence element; and
(b) a localization signal for directing transport of said polypeptide into a DNA containing organelle;
said nucleic acid construct actively transports into said DNA containing organelle.

11. The nucleic acid construct of claim 10, wherein said DNA containing organelle is selected from the group consisting of a nucleus, a chloroplast and a mitochondria.

12. The nucleic acid construct of claim 10, wherein said at least one nucleic acid binding domain is derived from a nucleic acid binding protein.

13. The nucleic acid construct of claim 12, wherein said nucleic acid binding protein is a transcription factor.

14. The nucleic acid construct of claim 10, further comprising an additional polynucleotide segment encoding an additional polypeptide.

15. The nucleic acid construct of claim 14, wherein said additional polynucleotide segment being positioned so as to be under a transcriptional control of said at least one nucleic acid sequence element.

16. The nucleic acid construct of claim 10, wherein said at least one nucleic acid sequence element is as set forth in SEQ ID NO:3.

17. The nucleic acid construct of claim 10, wherein said localization signal for directing transport of said polypeptide into said DNA containing organelle is selected from the group consisting of a nuclear localization signal (NLS), a mitochondrial localization signal (MLS) and a chloroplast localization signal (CLS).

18. The nucleic acid construct of claim 10, wherein said transport of said polypeptide into said DNA containing organelle is inducible via an extracellular stimulus.

19. The nucleic acid construct of claim 18, wherein said extracellular stimulus is selected from the group consisting of a growth factor stimulus, a cytokine stimulus, a hormone stimulus and a conditional stimulus.

20. The nucleic acid construct of claim 19, wherein said conditional stimulus is selected from the group consisting of a radiation, a temperature shift and a hypertonic treatment.

21. A method of facilitating active transport of a polynucleotide of interest into a DNA containing organelle of a eukaryotic cell, the method comprising:
(a) introducing into a cytoplasm of the eukaryotic cell a nucleic acid construct including the polynucleotide of interest and at least one nucleic acid sequence element; and
(b) providing within said cytoplasm of the eukaryotic cell a polypeptide including:
(i) a nucleic acid binding domain being capable of specifically binding said at least one nucleic acid sequence element;
(ii) a localization signal for directing transport of said polypeptide into the DNA containing organelle;
thereby facilitating active transport of the polynucleotide of interest into the DNA containing organelle of the eukaryotic cell.

22. The method of claim 21, wherein the DNA containing organelle is selected from the group consisting of a nucleus, a chloroplast and a mitochondria.

23. The method of claim 21, wherein the polynucleotide of interest encodes a polypeptide of interest.

24. The method of claim 23, wherein the polynucleotide of interest is positioned under a transcriptional control of said at least one nucleic acid sequence element.

25. The method of claim 21, wherein said at least one nucleic acid sequence element is as set forth in SEQ ID NO:3.

26. The method of claim 21, wherein said localization signal for directing transport of said polypeptide into the DNA containing organelle is selected from the group consisting of a nuclear localization signal (NLS), a mitochondrial localization signal (MLS) and a chloroplast localization signal (CLS).

27. The method of claim 21, wherein the step of providing within the eukaryotic cell said polypeptide is effected by a lipid based delivery system.

28. The method of claim 21, wherein said polypeptide is endogenous to the eukaryotic cell and further wherein the step of providing within the eukaryotic cell said polypeptide is effected by inducing expression or activity of said polypeptide.

29. The method of claim 21, wherein the step of providing within said eukaryotic cell said polypeptide is effected by introducing into said eukaryotic cell an additional nucleic acid construct capable of expressing said polypeptide.

30. A nucleic acid construct system comprising:
(a) a first nucleic acid construct including at least one nucleic acid sequence element; and
(b) a second nucleic acid construct including a polynucleotide segment encoding a polypeptide including:
(i) a nucleic acid binding domain being capable of specifically binding said at least one nucleic acid sequence element;
(ii) a localization signal for directing transport of said polypeptide into a DNA containing organelle;
such that when said first and said second nucleic acid constructs are introduced into a cell, expression of said polypeptide from said polynucleotide segment of said second nucleic acid construct directs transport of said first nucleic acid construct into said DNA containing organelle.

31. The nucleic acid construct system of claim 30, wherein said DNA containing organelle is selected from the group consisting of a nucleus, a chloroplast and a mitochondria.

32. The nucleic acid construct system of claim 30, wherein said nucleic acid binding domain is derived from a nucleic acid binding protein.

33. The nucleic acid construct system of claim 32, wherein said nucleic acid binding protein is a transcription factor.

34. A eukaryotic cell comprising the nucleic acid construct system of claim 30.

35. The nucleic acid construct system of claim 30, wherein said first nucleic acid construct further includes an additional polynucleotide segment encoding a polypeptide.

36. The nucleic acid construct system of claim 35, wherein said additional polynucleotide segment is positioned under a transcriptional control of said at least one nucleic acid element.

37. The nucleic acid construct system of claim 30, wherein said at least one nucleic acid sequence element is as set forth in SEQ ID NO:3.

38. The nucleic acid construct system of claim 30, wherein said localization signal for directing transport of said polypeptide into said DNA containing organelle is selected from the group consisting of a nuclear localization signal (NLS), a mitochondrial localization signal (MLS) and a chloroplast localization signal (CLS).

39. A method of detecting and isolating polynucleotides encoding polypeptides capable of binding a nucleic acid sequence element of interest, the method comprising:
(a) preparing a library of nucleic acid constructs each including:
(i) a first polynucleotide segment including at least one nucleic acid sequence element;
(ii) a second polynucleotide segment capable of generating reporter activity;
(iii) a third polynucleotide segment encoding a chimeric polypeptide including a distinct putative nucleic acid sequence binding domain and a localization signal for a DNA containing organelle;
(b) introducing said expression library into a plurality of eukaryotic cells; and
(c) screening for a cell or cells of said plurality of cells exhibiting a predetermined level or localization pattern of said reporter activity, thereby detecting and isolating polynucleotides encoding polypeptides capable of binding the nucleic acid sequence element of interest.

40. The method of claim 39, wherein said DNA containing organelle is selected from the group consisting of a nucleus, a chloroplast and a mitochondria.

41. The nucleic acid construct of claim 39, wherein said third polynucleotide segment is positioned under a transcriptional control of said at least one nucleic acid sequence element.

42. The method of claim 39, wherein said at least one nucleic acid sequence element is as set forth in SEQ ID NO:3.

43. The method of claim 39, wherein said localization signal for said DNA containing organelle is selected from the group consisting of a nuclear localization signal (NLS), a mitochondrial localization signal (MLS) and a chloroplast localization signal (CLS).

44. The method of claim 39, wherein said first polynucleotide region is positioned upstream or downstream of said second polynucleotide region.

45. A method of detecting and isolating nucleic acid sequence elements to which is bound a polypeptide of interest, the method comprising:
(a) preparing a library of nucleic acid constructs each including:
(i) a first polynucleotide segment including a distinct putative nucleic acid sequence element;
(ii) a second polynucleotide segment capable of generating reporter activity;
(iii) a third polynucleotide segment encoding a chimeric polypeptide including the polypeptide of interest and a localization signal for a DNA containing organelle;
(b) introducing said expression library into a plurality of eukaryotic cells; and
(c) screening for a cell or cells of said plurality of cells exhibiting a predetermined level or localization pattern of said reporter activity, thereby detecting and isolating nucleic acid sequence elements to which is bound the polypeptide of interest.

46. The method of claim 45, wherein said DNA containing organelle is selected from the group consisting of a nucleus, a chloroplast and a mitochondria.

47. The method of claim 45, wherein said localization signal for said DNA containing organelle is selected from the group consisting of a nuclear localization signal (NLS), a mitochondrial localization signal (MLS) and a chloroplast localization signal (CLS).

48. The method of claim 45, wherein said first polynucleotide region is positioned upstream or downstream of said second polynucleotide region.

49. The nucleic acid construct system of claim 45, wherein the polypeptide of interest is a transcription factor.


SEQUENCE LISTING (3 sequences)

SEQ ID NO: 1 - DNA, 112 bp, Artificial sequence
  5 X tandom repeats of kappa B motif constructed into a pGL3 vector
catgtctggatccaagctaggggactttccgcttggggactttccgctggggactttccgctggggactttccgctggggactttccgcggtgactctagagggggatcccg

SEQ ID NO: 2 - DNA, 88 bp, Artificial sequence
  Peptide nucleic acid (PNA) recognition sequence
cctctctctctctctctctctctctctctctctctctctctcggtacctctctctctctctctctctctctctctctctctctctctc

SEQ ID NO: 3 - DNA, 11 bp, Artificial sequence
  Kappa B motif
ggggactttcc